(19) United States
(12) Patent Application Publication
(10) Pub. No.: US 2004/0176790 A1
(43) Pub. Date: Sep. 9, 2004

(54) Single lumen catheter shaft for a balloon catheter

(75) Inventor: James Alexus Coyle, Somerville, MA (US)

Correspondence Address: MEDTRONIC VASCULAR, INC., IP LEGAL DEPARTMENT, 3576 UNOCAL PLACE, SANTA ROSA, CA, 95403, US

(73) Assignee: Medtronic AVE, Inc.

(21) Appl. No.: 10/376,288

(22) Filed: Mar. 3, 2003

(51) Int. Cl. A61M 29/00

(52) U.S. Cl. 606/194

(57) ABSTRACT

A balloon catheter includes a one-piece shaft and a guidewire track for use of a guidewire that runs along an outer surface of the one-piece shaft adjacent to a longitudinal central axis of the one-piece shaft. The one-piece shaft has an inflation lumen formed therein. The one-piece shaft is formed to include a substantially C-shaped cross section, which has two endpoints and forms the guidewire track. The dimensions of the wall thicknesses between the inflation lumen and the guidewire track may be varied to help maintain the guidewire track and the gap between the endpoints of the C-shaped cross section such that a guidewire is maintained on the guidewire track and does not traverse the gap. In addition, a reinforcement may be included in the single lumen shaft to maintain the integrity of the guidewire track.

### BACKGROUND OF THE INVENTION

1. Field of the Invention

The present invention relates to a medical device. More specifically, the invention relates to a balloon catheter that has, a single lumen shaft with a substantially C-shaped cross section.

2. Background Art

Cardiovascular disease, including atherosclerosis, is the leading cause of death in the U.S. One method for treating atherosclerosis and other forms of coronary narrowing is percutaneous transluminal coronary angioplasty, commonly referred to as “angioplasty” or “PTCA”. The objective in angioplasty is to enlarge the lumen of the affected coronary artery by radial hydraulic expansion. The procedure is accomplished by inflating a balloon of a balloon catheter within the narrowed lumen of the coronary artery. Radial expansion of the coronary artery occurs in several different dimensions, and is related to the nature of the plaque. Soft, fatty plaque deposits are flattened by the balloon, while hardened deposits are cracked and split to enlarge the body lumen.

Two types of catheters commonly used in angioplasty procedures are referred to as over-the-wire (OTW) catheters and rapid exchange (RX) catheters. An OTW catheter's guidewire lumen runs the entire length of the catheter and is attached to, or enveloped within, an inflation lumen. Thus, the entire length of an OTW catheter is tracked over a guidewire during a PTCA procedure. A RX catheter, on the other hand, has a guidewire lumen that extends within only the distalmost portion of the catheter. Thus during a PTCA procedure only the distalmost portion of a RX catheter is tracked over a guidewire.

Both OTW and RX catheters have advantages. There are instances when the guidewire and not the catheter must be replaced. For example, the guidewire may become damaged during the procedure or it may be discovered during the procedure that a different shape, length, or size of guidewire is needed. The OTW catheter allows for easy guidewire exchange. The OTW catheter may remain in place while the guidewire is removed, replaced, and simply slid back past the treatment site using the existing OTW catheter. However, there are instances when the catheter and not the guidewire must be replaced. For example, the catheter may become damaged during the procedure, or it may be discovered during the procedure that a different size balloon is necessary to enlarge the body lumen. The RX catheter allows for easy catheter exchange. The guidewire may remain in place while the RX catheter is removed, replaced, and simply slid back over the existing guidewire to the treatment site.

Both OTW and RX catheters have dual lumens, a guidewire lumen and an inflation lumen, for at least a portion of their length. In a catheter employing two shafts to create the dual lumens, there is necessarily a bond area that attaches the two shafts. The bond area may be weak and therefore make the catheter undesirable or inoperable. Another problem associated with the bond area is that it may be too large, and therefore make the catheter inoperable. A single lumen shaft reduces these problems by simplifying the bond area and/or bonding process.

It is therefore an object of the present invention to provide a catheter with a single lumen shaft in order to simplify the bond area that occurs between two shafts in a dual lumen catheter. The overall simplicity of the design is an object of the present invention. It is another object of the present invention to provide a single lumen shaft that may be utilized in a catheter to be operated in a manner similar to that of an OTW catheter or a RX catheter.

### BRIEF SUMMARY OF THE INVENTION

To achieve the foregoing and other objects, and in accordance with the purposes of the present invention as embodied and broadly described herein, the balloon catheter of the present invention provides a balloon catheter with a single lumen shaft with a substantially C-shaped cross section. The balloon catheter is comprised of a single lumen shaft with a substantially C-shaped cross section, a guidewire track that runs along an outer surface of the single lumen shaft adjacent to a longitudinal central axis of the catheter for use of a guidewire, and a balloon.

The present invention can form the basis of a stent delivery system, as well as an angioplasty catheter. In the present invention, the balloon catheter has a one-piece single lumen shaft with an inflation lumen formed therein. The single lumen shaft is extruded to form a substantially C-shaped cross section. The C-shaped cross-section has two endpoints and forms a guidewire track that runs adjacent to the longitudinal central axis of the catheter. The guidewire track is approximately 0.016 inches wide. The guidewire track runs along an outer surface of the single lumen shaft adjacent to a longitudinal central axis of the single lumen shaft. Accordingly, the guidewire track allows the catheter to be tracked over a guidewire to the treatment site. A gap approximately of 0.003 inches is maintained between the two endpoints of the C-shaped cross section. The gap remains at such a width to ensure that the guidewire does not traverse the gap. Therefore, the guidewire is contained on the guidewire track while the catheter is tracked over the guidewire to the treatment site.

Further, a balloon is mounted on a distal end of the catheter. The balloon may be of any shape or size customarily used in procedures such as angioplasty and stent delivery. The inflation lumen is in fluid communication with the balloon so that the balloon may be inflated. The balloon is disposed on a distal end of the single lumen shaft.

The single lumen shaft is of sufficient wall thickness to avoid deforming during high pressure balloon inflations. The wall thickness of the single lumen shaft provides sufficient strength to maintain the guidewire track and the gap between the endpoints, as well as allowing for overall flexibility of the catheter. In addition, the dimensions of the wall thicknesses between the inflation lumen and the guidewire track may be varied to help maintain the guidewire track and the gap between the endpoints of the C-shaped cross section.

A guidewire track according to the present invention allows for a catheter to be tracked over a guidewire to the treatment site. Thus, a single lumen shaft according to the present invention can extend for only a distal portion of the catheter, forming a rapid-exchange-type catheter, or an entire length of the catheter, forming an over-the-wire type catheter.

When the single lumen shaft extends for only the distal portion of the catheter, the catheter includes a transition section. In this embodiment, a single lumen hypotube extends from a proximal end of the catheter to a proximal end of the transition section. The single lumen shaft includes a guidewire track that extends from a distal end of the transition section to a proximal end of the balloon. For the relatively short length of the transition section, there are two lumens while at all other points along the length of the catheter shaft there is only a single lumen. The transition section includes a transition shaft, a guidewire shaft and a proximal guidewire port. The guidewire port accommodates entry and exit of the guidewire to the guidewire track such that the catheter is trackable over the guidewire to the treatment site.

In an alternate embodiment of the present invention, a reinforcement is disposed within the single lumen shaft to help maintain the guidewire track and the gap between the endpoints of the C-shaped cross section. In one embodiment, the reinforcement may be a braided mesh reinforcement constructed of NITINOL, or any other suitable material. The single lumen shaft may be extruded around the reinforcement, such that the reinforcement substantially surrounds the guidewire track and is positioned adjacent to the inflation lumen. Alternatively or in addition to the use of a reinforcement, the dimensions of the wall thicknesses between the inflation lumen and the guidewire track may be varied to help maintain the guidewire track and the gap between the endpoints of the C-shaped cross section.

### BRIEF DESCRIPTION OF THE DRAWINGS/FIGURES

The foregoing and other features and advantages of the invention will be apparent from the following, more particular description of a preferred embodiment of the invention, as illustrated in the accompanying drawings.

FIG. 1 is a side elevational view of a stent delivery system incorporating the present invention.

FIG. 2 is an enlarged view of a transition section 112 of the catheter in FIG. 1.

FIG. 3 is a cross-sectional view of the single lumen shaft of the catheter of FIG. 1 taken along line A-A.

FIG. 4 is a side elevational view of a stent delivery system incorporating the present invention in an over-the-wire type catheter.

FIG. 5 is a cross-sectional view of an alternate embodiment of the single lumen shaft of the catheter of FIG. 1 taken along line A-A.

FIG. 6 is an alternate embodiment of the cross-section of the single lumen shaft shown in FIG. 5.

### DETAILED DESCRIPTION OF THE INVENTION

An embodiment of the present invention is now described with reference to the figures, where like reference numbers indicate identical or functionally similar elements. Also in the figures, the left most digit of each reference number corresponds to the figure in which the reference number is first used. While specific configurations and arrangements are discussed, it should be understood that this is done for illustrative purposes only. A person skilled in the relevant art will recognize that other configurations and arrangements can be used without departing from the spirit and scope of the invention.

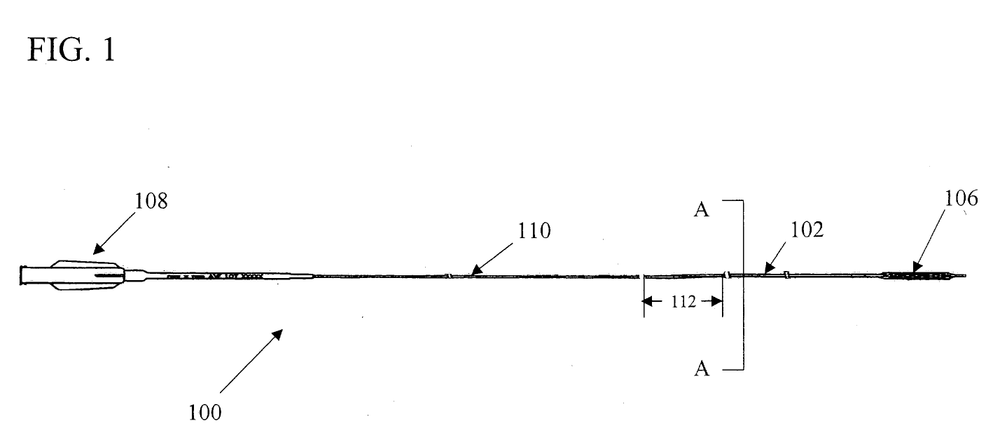

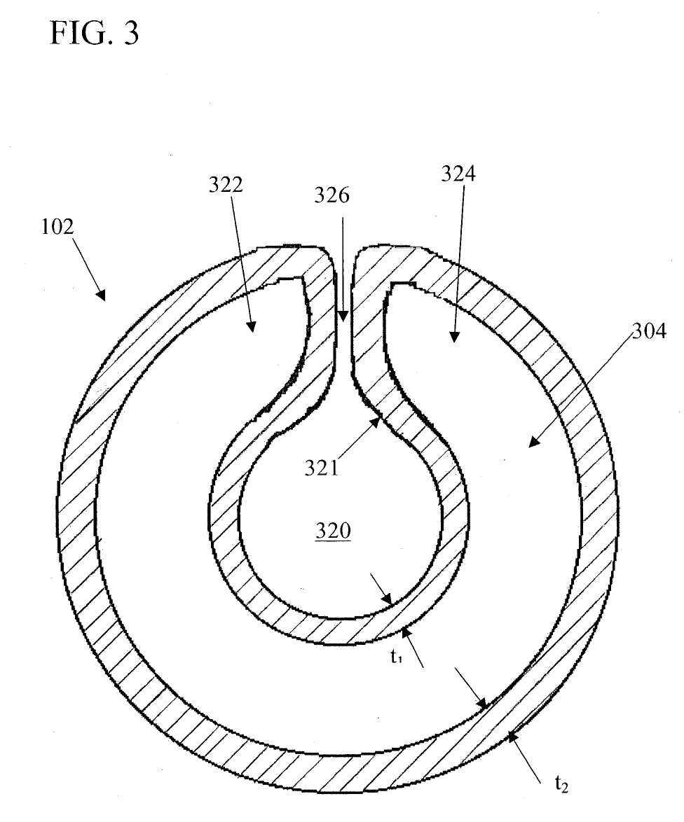

Referring to FIGS. 1 and 3, an embodiment of a stent delivery system utilizing a catheter according to the present invention is shown. A balloon catheter 100 is provided with a single lumen shaft 102, wherein inflation lumen 304 is formed therein. Single lumen shaft 102 is a one-piece shaft that has a substantially C-shaped cross section, as shown in FIG. 3. The C-shaped cross section forms a guidewire track 320 sized to accommodate a guidewire. In one embodiment, guidewire track 320 is approximately 0.016 inches wide. Guidewire track 320 runs along an outer surface 321 of single lumen shaft 102 adjacent to a longitudinal central axis of single lumen shaft 102. Guidewire track 320 allows the catheter to be tracked over a guidewire to the treatment site.

The C-shaped cross section has two endpoints 322 and 324, and a gap 326 that is substantially maintained between the endpoints 322 and 324. In one embodiment, gap 326 is approximately 0.003 inches wide, and remains at such a width to contain a guidewire on guidewire track 320. Gap 326 remains at such a width to ensure that the guidewire does not traverse gap 326. Therefore, the guidewire is contained on guidewire track 320 while the catheter is tracked over the guidewire to the treatment site.

At a proximal end of catheter 100, a hub 108 is connected to and in fluid communication with inflation lumen 304 via a hypotube or a proximal shaft. Mounted at a distal end of catheter 100 is an inflatable balloon 106. Inflation lumen 304 is in fluid communication with an interior of balloon 106. Hub 108 is preferably a luer fitting that allows inflation means (not shown) to be connected thereto for inflation of balloon 106.

Balloon 106 may be heat welded or glued to the distal portion of catheter 100. Balloon 106 can be any appropriate size or shape, and any material, which is relatively elastic and deformable. Non-exhaustive examples for balloon 106 include polymers such as polyethylene, polyethylene terephalate (PET), nylon, polyurethane, and polyether block amide, which is commonly referred to under the trademark PEBAX.

Single lumen shaft 102 is of sufficient wall thicknesses and made of any appropriate polymeric material to avoid deforming during high pressure balloon inflations. In addition, the dimensions of the wall thicknesses, shown on FIG. 3 as t₁ and t₂, may be varied to help maintain gap 326. The material used for single lumen shaft 102 should be of sufficient strength to maintain guidewire track 320 and gap 326, as well as allowing for overall flexibility of catheter 100. Possible materials used in construction of the shaft include: PET, nylon, polyethylene, PEBAX, composites, or any combination of the above mentioned materials.

Shaft 102 maybe manufactured from a solid tube of material by extrusion processes generally well known in the art. One method of manufacture is that a solid tube is extruded over appropriately shaped support mandrels, or wires, to form inflation lumen 304, guidewire track 320, and gap 326. Inflation lumen 304 may be made in various suitable shapes or sizes. The support mandrels, or wires, may then be removed, resulting in one-piece single lumen shaft 102. The resulting one-piece single lumen shaft 102 is then heat treated into its final desired shape. Another method of manufacture is that the shaft can be extruded directly into a desired shape by one skilled in the art.

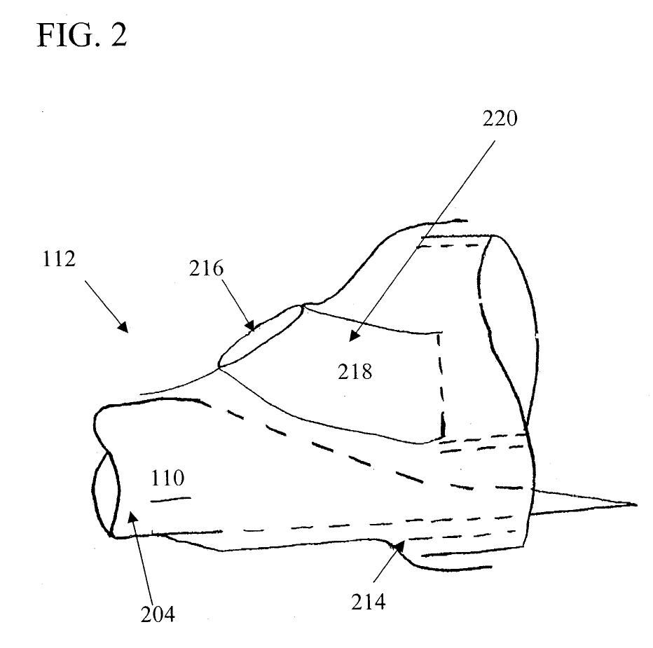

Single lumen shaft 102 may be used in an OTW-type catheter or a RX-type catheter. An embodiment of the present invention used in a RX-type catheter is shown in FIGS. 1-3. In a RX-type catheter, single lumen shaft 102 extends for only a distal portion of catheter 100. A transition section 112 includes a circular transition shaft 214, a guidewire shaft 218, and a proximal guidewire port 216. Guidewire shaft 218 forms a guidewire lumen 220 that is in communication with guidewire track 320 such that a guidewire backloaded into catheter 100 at a distal end thereof exits via proximal guidewire port 216.

A proximal portion 110 of catheter 100, in this embodiment an inflation shaft preferably comprised of a hypotube, is attached to a proximal end of transition shaft 214. Single lumen shaft 102 is attached to a distal end of transition shaft 214 such that an inflation lumen 204 of proximal portion 110 is in fluid communication with inflation lumen 304 of single lumen shaft 102. In another embodiment, proximal portion 110 of catheter 100 may be formed from a reinforced polymeric tube.

In order to be utilized in a manner similar to a RX catheter, a guidewire is tracked to the treatment site and then backloaded into a distal end of catheter 100 onto guidewire track 320. The catheter is then tracked over the guidewire such that an operator can maintain contact with the guidewire, which proximately exits the catheter via proximal guidewire port 216, throughout the procedure. In this embodiment of the present invention, to exchange catheters during use, catheter 100 is removed while the guidewire stays in place, such that a new catheter may be tracked over the guidewire to the treatment site.

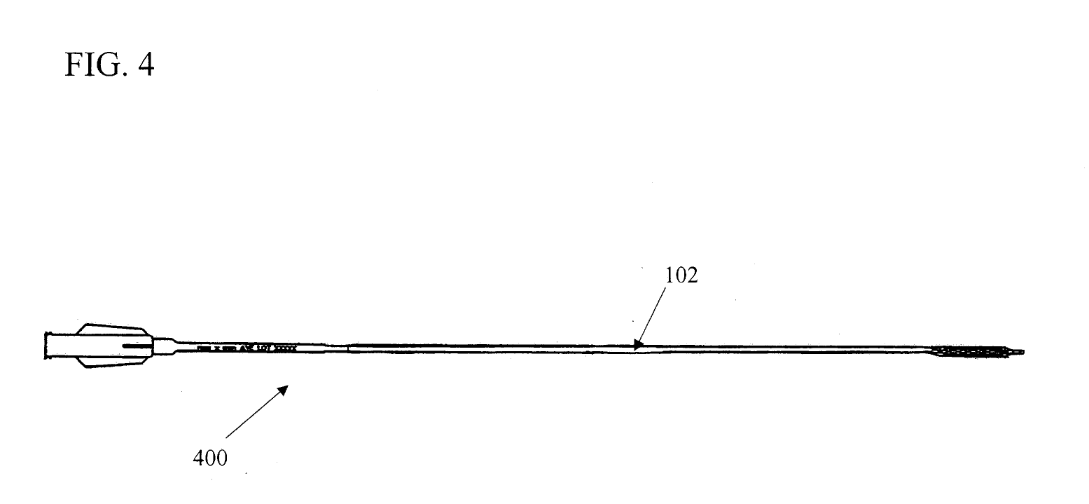

An embodiment of the present invention that may be used in a manner similar to an OTW catheter is shown in FIG. 4. In catheter 400, single lumen shaft 102 runs the entire length of catheter 400 allowing the catheter to be tracked over a guidewire contained within guidewire track 320. In this embodiment, a guidewire may be exchanged while catheter 400 is left in place once the old guidewire is removed.

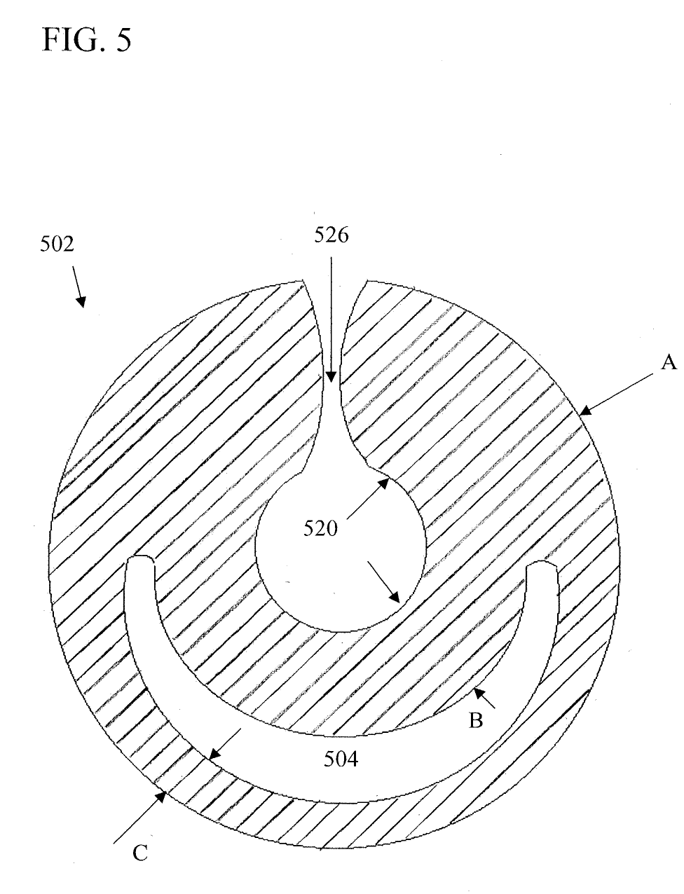

FIG. 5 shows an alternate embodiment of single lumen shaft 102 shown in FIG. 3. A single lumen shaft 502 is substantially C-shaped with an inflation lumen 504 formed therein. The dimensions of the wall thicknesses, shown in FIG. 5 as dimensions A, B, and C, may be varied to maintain the desired C-shaped cross section of single lumen shaft 502. The shape of the shaft and the values for dimensions A, B and C should be chosen so as to ensure that: inflation lumen 504 may withstand inflation pressures up to 20 ATM without deforming in shape; guidewire track 520 will not deform significantly as single lumen shaft 502 is tracked to the treatment site; gap 526 will not open up to the extent that a standard 0.014″ guide wire can pass therethrough; and single lumen shaft 502 has significant flexibility so that as it is being tracked to the treatment site there is not excessive resistance in advancing it.

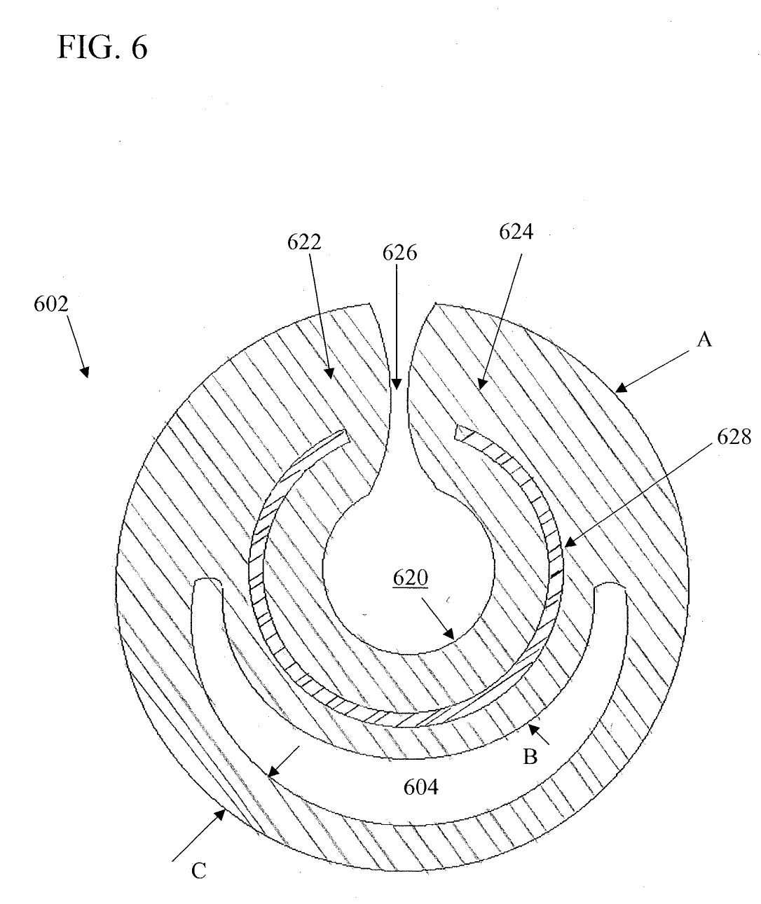

FIG. 6 shows an alternate embodiment of single lumen shaft 502 shown in FIG. 5. Alternatively or in addition to varying the dimensions A, B, and C of the wall thicknesses to maintain the desired C-shaped cross section of single lumen shaft 602, a reinforcement 628 may be included in single lumen shaft 602 to help maintain guidewire track 620 and gap 626 between endpoints 622 and 624. Reinforcement 628 substantially surrounds guidewire track 620 and is positioned above inflation lumen 604. Reinforcement 628 helps ensure that gap 626 remains at such a width to ensure that the guidewire does not traverse gap 626. Therefore, the guidewire is contained on guidewire track 620 while the catheter is tracked over the guidewire to the treatment site. In one embodiment, reinforcement 628 may be of a braided mesh comprised of NITINOL, or any other suitable elastic material. NITINOL is desirable because it can undergo deformations when under the influence of force, but then spring back to its original shape after the force is removed.

Shaft 602 may be manufactured by extrusion processes generally well known in the art. A solid polymer tube is extruded over an appropriately shaped support mandrel, or wire, to form guidewire track 620 and a portion of gap 626. Reinforcement 628, that may be heat set into a substantially C-shaped cross-section, is then slid over the extruded plastic tube. Additional material is then extruded over reinforcement 628 and an additional mandrel or wire for forming inflation lumen 604, such that gap 626 is maintained. The support mandrels, or wires, may then be removed, resulting in a one-piece single lumen shaft. The shaft is then heat treated to set its shape.

While this invention has been particularly shown and described with reference to preferred embodiments thereof, it will be understood by those skilled in the art that various changes in form and details may be made therein without departing from the spirit and scope of the invention.

What is claimed is:

1. A balloon catheter comprising:
a one-piece shaft having an inflation lumen formed therein, wherein the shaft has a substantially C-shaped cross-section with two endpoints such that a guidewire track runs along an outer surface of the one-piece shaft adjacent to a longitudinal central axis of the one-piece shaft and a gap is maintained between the two endpoints; and
a balloon disposed on a distal end of the one-piece shaft, wherein an interior of the balloon is in fluid communication with the inflation lumen.

2. The balloon catheter of claim 1, wherein the one-piece shaft is formed by extrusion.

3. The balloon catheter of claim 1, wherein the one-piece shaft extends only through a distal portion of the catheter to create a rapid-exchange type catheter.

4. The balloon catheter of claim 3, further comprising:
a transition section having a proximal guidewire port for access to a guidewire within the guidewire track.

5. The balloon catheter of claim 1, wherein the one-piece shaft extends for substantially the full length of the catheter to create an over-the-wire type catheter.

6. The balloon catheter of claim 1, wherein the gap is approximately 0.003 inches wide.

7. The balloon catheter of claim 1, wherein the guidewire track is approximately 0.016 inches in diameter.

8. The balloon catheter of claim 1, wherein the inflation lumen is in fluid communication with a hub mounted on a proximal end of the catheter.

9. A balloon catheter comprising:
a one-piece shaft having an inflation lumen formed therein, wherein the shaft has a substantially C-shaped cross-section with two endpoints such that a guidewire track runs along an outer surface of the one-piece shaft adjacent to a longitudinal central axis of the one-piece shaft and a gap is maintained between the two endpoints;
a reinforcement located within the shaft, wherein the reinforcement maintains the C-shaped cross-section of the one-piece shaft; and
a balloon disposed on a distal end of the one-piece shaft, wherein an interior of the balloon is in fluid communication with the inflation lumen.

10. The balloon catheter of claim 9, wherein the one-piece shaft is formed through extrusion.

11. The balloon catheter of claim 10, wherein the shaft extends for only a distal portion of the catheter to create a rapid-exchange type catheter.

12. The balloon catheter of claim 12, further comprising:
a transition section having a proximal guidewire port for access to a guidewire within the guidewire track.

13. The balloon catheter of claim 9, wherein the one-piece shaft extends for substantially the full length of the catheter to create an over-the-wire type catheter.

14. The balloon catheter of claim 9, wherein the gap is approximately 0.003 inches wide.

15. The balloon catheter of claim 9, wherein the guidewire track is approximately 0.016 inches in diameter.

16. The balloon catheter of claim 9, wherein the inflation lumen is in fluid communication with a hub mounted on a proximal end of the catheter.

17. The balloon catheter of claim 9, wherein the reinforcement is comprised of a braided mesh made of NITINOL.
